(19) United States
(12) Patent Application Publication
(10) Pub. No.: US 2013/0204153 A1
(43) Pub. Date: Aug. 8, 2013

(54) GENERATING AN ALARM BASED ON BRAIN WAVE PATTERNS OF A USER

(71) Applicant: Emily Ruth Buzhardt, Nashville, TN (US)

(72) Inventor: Emily Ruth Buzhardt, Nashville, TN (US)

(21) Appl. No.: 13/367,060

(22) Filed: Feb. 6, 2012

(51) Int. Cl. G08B 23/00; A61B 5/0476

(52) U.S. Cl. 600/544; 340/575

(57) ABSTRACT

A method and a device are disclosed including detecting a brainwave pattern associated with a particular mental and/or physical state of a user, classifying the detected brainwave pattern to identify the particular mental/physical state of the user, and generating an alarm signal to notify the user, other authority, and/or to be used otherwise. In various embodiments, the brainwave pattern data may be recorded and used for statistical analysis, work or process scheduling, or other management decisions. In various embodiments, the brainwave pattern is detected and/or recorded using a cap or harness with embedded electrodes, a controller for controlling the electrodes, collecting data, transmitting the data to a local or central data storage device, and generating an alarm.

### TECHNICAL FIELD

This application relates generally to safety. More specifically, this application relates to issuing an alert based on a user's brain wave patterns such as pre-sleep brain wave patterns.

### BRIEF DESCRIPTION OF THE DRAWINGS

The drawings, when considered in connection with the following description, are presented for the purpose of facilitating an understanding of the subject matter sought to be protected.

FIG. 1 shows an embodiment of a network computing environment wherein the disclosure may be practiced;

FIG. 2 shows an embodiment of a computing device that may be used in the network computing environment of FIG. 1;

FIG. 3 shows an example operating environment wherein the disclosure may be practiced;

FIG. 4A shows an example arrangement for recording brainwave patterns;

FIG. 4B-4D show examples of brainwave patterns associated with successive pre-sleep states;

FIG. 5A shows an example brainwave detection cap and controller; and

FIG. 5B shows an example brainwave detection cap installed in the cab of a vehicle.

### DETAILED DESCRIPTION

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while following description references particular brain wave patterns like pre-sleep patterns, it will be appreciated that the disclosure may be used with other types of brain wave patterns such as those associated with hallucination, intoxication, seizure and the like.

Briefly described, a device and a method are disclosed including detecting a brainwave pattern associated with a particular mental and/or physical state of a user, classifying the detected brainwave pattern to identify the particular mental/physical state of the user, and generating an alarm signal to notify the user, other authority, and/or to be used otherwise. In various embodiments, the brainwave pattern data may be recorded and used for statistical analysis, work or process scheduling, or other management decisions. In various embodiments, the brainwave pattern is detected and/or recorded using a cap or harness with embedded electrodes, a controller for controlling the electrodes, collecting data, transmitting the data to a local or central data storage device, and generating an alarm.

In many industrial settings and other situations where people operate machinery or otherwise have to be alert to perform a task, mental alertness and acuity is highly needed. Various mental states and/or physical conditions, such as sleepiness, drug-induced or otherwise caused hallucination, narcoses, inebriation, intoxication, brain seizure, epileptic attack, and the like may adversely affect mental alertness causing an operator to lose control resulting in bodily injury, property damage, or other damages. Many industrial machinery have safety switches, required to be pressed during normal operation, which are triggered by omission of the operator when the operator loses control, such as when the operator's hand or feet are taken off the safety switch. However, such measures may not work in every situation. For example, if a truck driver on an interstate route falls asleep, the truck moving at 60 miles per hour cannot be safely and automatically stopped and pulled off the road by a safety switch. A method of detection of such mental states is highly desirable to alert the operator to take some appropriate action, such as stopping and pulling over the truck in the foregoing example.

Electroencephalography (EEG) is the recording of brain electrical signals detected by electrodes located in close proximity of the scalp. EEG measures voltage variations resulting from electrical current flowing through the neurons (nerve cells) of the brain. In medical practice, EEG is generated over a period of time, usually 20-40 minutes long, from electrodes placed on the scalp. In neurology, one application of EEG is for epilepsy, which shows clear abnormalities on the recorded waveform. EEG may also be used for the diagnosis of stroke and other brain disorders. Despite limited spatial resolution, EEG is a valuable tool for detection and diagnosis, especially when millisecond-range temporal resolution is needed.

Other brainwave detection techniques derived from EEG include Evoked Potentials (EP), which involves averaging the EEG activity time-locked between bounds of a presentation of a stimulus of some sort, such as visual, somatosensory, or auditory stimulation; and Event-Related Potentials (ERPs), which is the averaged EEG responses that are time-locked to more complex processing of stimuli, such as cognitive and psycho-physiological activities.

EEG shows oscillations at different frequencies. Some of these oscillations have characteristic frequency ranges, spatial distributions, and are associated with different states of brain's function, such as waking state and the various sleep stages.

To generate EEG, the electrodes are typically placed on the scalp with a conductive gel or paste. Many systems typically use electrodes, each of which is attached to an individual wire. Some systems use caps or nets into which electrodes are embedded, which is particularly useful when high-density arrays of electrodes are needed.

One of the most common causes of accidents or loss of control during operation of machinery or vehicles, is getting drowsy and/or falling asleep. Sleep may be divided into two broad types: Rapid Eye Movement (REM) and Nonrapid Eye Movement (NREM) sleep. Based on EEG, NREM may be divided further into four stages I, II, III, and IV. NREM and REM occur in alternating cycles, each lasting approximately 90-100 minutes, with a total of 4-6 cycles. In general, in the healthy young adult NREM sleep accounts for 75-90% of sleep time (3-5% stage I, 50-60% stage II, and 10-20% stages III and IV). REM sleep accounts for 10-25% of sleep time.

The EEG waveforms associated with different brain states may be used to detect the current state of the brain and to identify any anomalies such as beginning to fall asleep.

### Illustrative Operating Environment

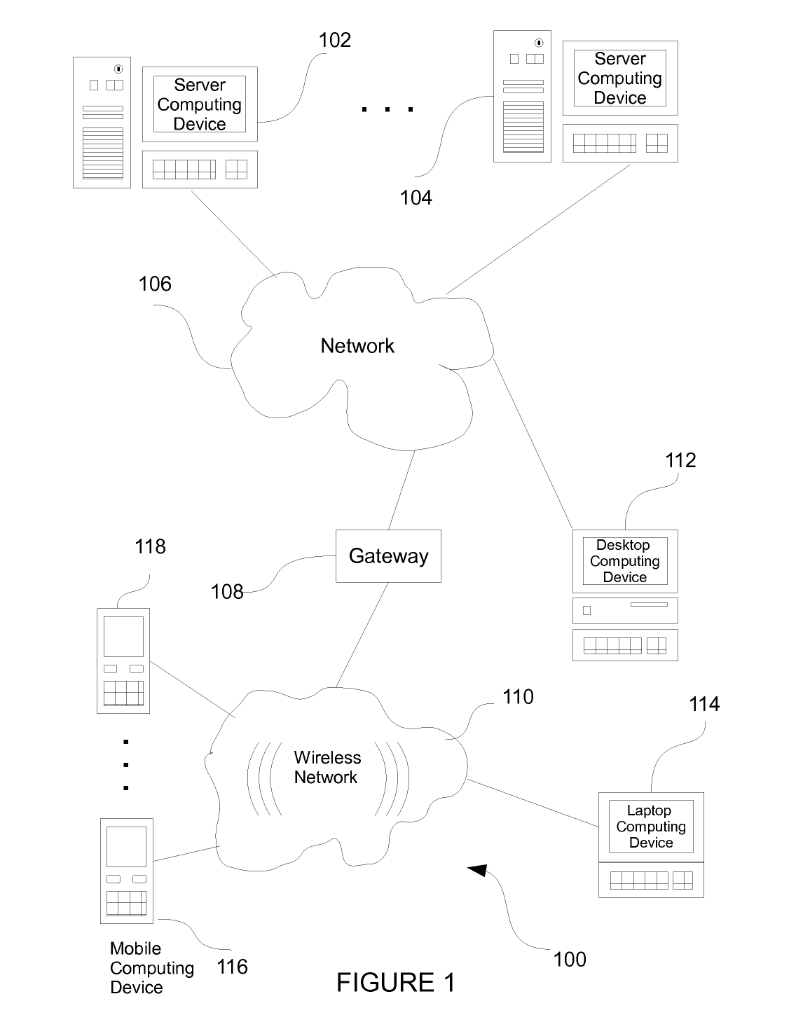

FIG. 1 shows components of an illustrative environment in which the disclosure may be practiced. Not all the shown components may be required to practice the disclosure, and variations in the arrangement and type of the components may be made without departing from the spirit or scope of the disclosure. System 100 may include Local Area Networks (LAN) and Wide Area Networks (WAN) shown collectively as Network 106, wireless network 110, gateway 108 configured to connect remote and/or different types of networks together, client computing devices 112-118, and server computing devices 102-104.

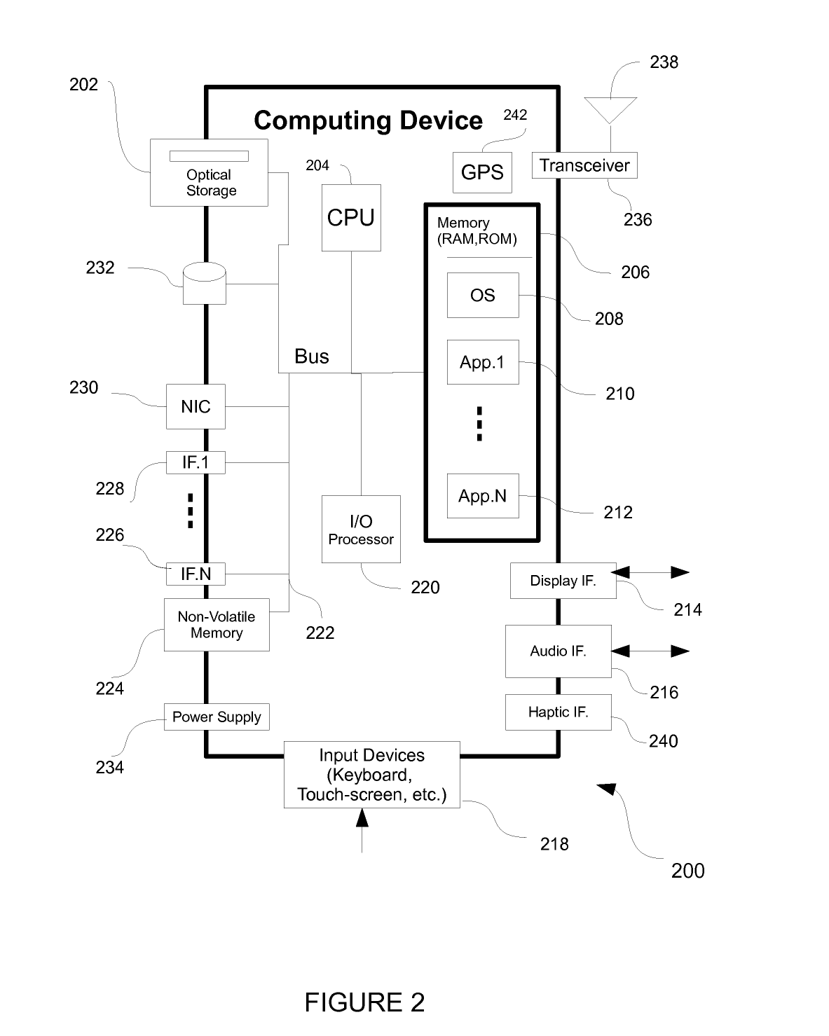

One embodiment of a computing device usable as one of client computing devices 112-118 is described in more detail below with respect to FIG. 2. Briefly, however, client computing devices 112-118 may include virtually any device capable of receiving and sending a message over a network, such as wireless network 110, or the like. Such devices include portable devices such as, cellular telephones, smart phones, digital cameras, display pagers, radio frequency (RF) devices, music players, digital cameras, infrared (IR) devices, Personal Digital Assistants (PDAs), handheld computers, laptop computers, wearable computers, tablet computers, integrated devices combining one or more of the preceding devices, and the like. Client device 112 may include virtually any computing device that typically connects using a wired communications medium such as personal computers, multiprocessor systems, microprocessor-based or programmable consumer electronics, network PCs, or the like. In one embodiment, one or more of client devices 112-118 may also be configured to operate over a wired and/or a wireless network.

Client devices 112-118 typically range widely in terms of capabilities and features. For example, a cell phone may have a numeric keypad and a few lines of monochrome LCD display on which only text may be displayed. In another example, a web-enabled client device may have a touch sensitive screen, a stylus, and several lines of color LCD display in which both text and graphic may be displayed.

A web-enabled client device may include a browser application that is configured to receive and to send web pages, web-based messages, or the like. The browser application may be configured to receive and display graphic, text, multimedia, or the like, employing virtually any web based language, including a wireless application protocol messages (WAP), or the like. In one embodiment, the browser application may be enabled to employ one or more of Handheld Device Markup Language (HDML), Wireless Markup Language (WML), WMLScript, JavaScript, Standard Generalized Markup Language (SMGL), HyperText Markup Language (HTML), eXtensible Markup Language (XML), or the like, to display and send information.

Client computing devices 12-118 also may include at least one other client application that is configured to receive content from another computing device, including, without limit, server computing devices 102-104. The client application may include a capability to provide and receive textual content, multimedia information, or the like. The client application may further provide information that identifies itself, including a type, capability, name, or the like. In one embodiment, client devices 112-118 may uniquely identify themselves through any of a variety of mechanisms, including a phone number, Mobile Identification Number (MIN), an electronic serial number (ESN), mobile device identifier, network address, such as IP (Internet Protocol) address, Media Access Control (MAC) layer identifier, or other identifier. The identifier may be provided in a message, or the like, sent to another computing device.

Client computing devices 112-118 may also be configured to communicate a message, such as through email, Short Message Service (SMS), Multimedia Message Service (MMS), instant messaging (IM), internet relay chat (IRC), Mardam-Bey's IRC (mIRC), Jabber, or the like, to another computing device. However, the present disclosure is not limited to these message protocols, and virtually any other message protocol may be employed.

Client devices 112-118 may further be configured to include a client application that enables the user to log into a user account that may be managed by another computing device. Such user account, for example, may be configured to enable the user to receive emails, send/receive IM messages, SMS messages, access selected web pages, download scripts, applications, or a variety of other content, or perform a variety of other actions over a network. However, managing of messages or otherwise accessing and/or downloading content, may also be performed without logging into the user account. Thus, a user of client devices 112-118 may employ any of a variety of client applications to access content, read web pages, receive/send messages, or the like. In one embodiment, for example, the user may employ a browser or other client application to access a web page hosted by a Web server implemented as server computing device 102. In one embodiment, messages received by client computing devices 112-118 may be saved in non-volatile memory, such as flash and/or PCM, across communication sessions and/or between power cycles of client computing devices 112-118.

Wireless network 110 may be configured to couple client devices 114-118 to network 106. Wireless network 110 may include any of a variety of wireless sub-networks that may further overlay stand-alone ad-hoc networks, and the like, to provide an infrastructure-oriented connection for client devices 114-118. Such sub-networks may include mesh networks, Wireless LAN (WLAN) networks, cellular networks, and the like. Wireless network 110 may further include an autonomous system of terminals, gateways, routers, and the like connected by wireless radio links, and the like. These connectors may be configured to move freely and randomly and organize themselves arbitrarily, such that the topology of wireless network 110 may change rapidly.

Wireless network 110 may further employ a plurality of access technologies including 2nd (2G), 3rd (3G), 4^{th} (4G), generation and any future generation technologies for radio access for cellular systems, WLAN, Wireless Router (WR) mesh, and the like. Access technologies such as 3G, 4G, and future access networks may enable wide area coverage for mobile devices, such as client devices 114-118 with various degrees of mobility. For example, wireless network 110 may enable a radio connection through a radio network access such as Global System for Mobil communication (GSM), General Packet Radio Services (GPRS), Enhanced Data GSM Environment (EDGE), WEDGE, Bluetooth, High Speed Downlink Packet Access (HSDPA), Universal Mobile Telecommunications System (UMTS), Wi-Fi, Zigbee, Wideband Code Division Multiple Access (WCDMA), and the like. In essence, wireless network 110 may include virtually any wireless communication mechanism by which information may travel between client devices 102-104 and another computing device, network, and the like.

Network 106 is configured to couple one or more servers depicted in FIG. 1 as server computing devices 102-104 and their respective components with other computing devices, such as client device 112, and through wireless network 110 to client devices 114-118. Network 106 is enabled to employ any form of computer readable media for communicating information from one electronic device to another. Also, network 106 may include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another.

Communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. Furthermore, remote computers and other related electronic devices could be remotely connected to either LANs or WANs via a modem and temporary telephone link. Network 106 may include any communication method by which information may travel between computing devices. Additionally, communication media typically may enable transmission of computer-readable instructions, data structures, program modules, or other types of content, virtually without limit. By way of example, communication media includes wired media such as twisted pair, coaxial cable, fiber optics, wave guides, and other wired media and wireless media such as acoustic, RF, infrared, and other wireless media.

### Illustrative Computing Device Configuration

FIG. 2 shows an illustrative computing device 200 that may represent any one of the server and/or client computing devices shown in FIG. 1. A computing device represented by computing device 200 may include less or more than all the components shown in FIG. 2 depending on the functionality needed. For example, a mobile computing device may include the transceiver 236 and antenna 238, while a server computing device 102 of FIG. 1 may not include these components. Those skilled in the art will appreciate that the scope of integration of components of computing device 200 may be different from what is shown. As such, some of the components of computing device 200 shown in FIG. 2 may be integrated together as one unit. For example, NIC 230 and transceiver 236 may be implemented as an integrated unit. Additionally, different functions of a single component may be separated and implemented across several components instead. For example, different functions of I/O processor 220 may be separated into two or more processing units.

With continued reference to FIG. 2, computing device 200 includes optical storage 202, Central Processing Unit (CPU) 204, memory module 206, display interface 214, audio interface 216, input devices 218, Input/Output (I/O) processor 220, bus 222, non-volatile memory 224, various other interfaces 226-228, Network Interface Card (NIC) 320, hard disk 232, power supply 234, transceiver 236, antenna 238, haptic interface 240, and Global Positioning System (GPS) unit 242. Memory module 206 may include software such as Operating System (OS) 208, and a variety of software application programs 210-212. Computing device 200 may also include other components not shown in FIG. 2. For example, computing device 200 may further include an illuminator (for example, a light), graphic interface, and portable storage media such as USB drives. Computing device 200 may also include other processing units, such as a math co-processor, graphics processor/accelerator, and a Digital Signal Processor (DSP).

Optical storage device 202 may include optical drives for using optical media, such as CD (Compact Disc), DVD (Digital Video Disc), and the like. Optical storage devices 202 may provide inexpensive ways for storing information for archival and/or distribution purposes.

Central Processing Unit (CPU) 204 may be the main processor for software program execution in computing device 200. CPU 204 may represent one or more processing units that obtain software instructions from memory module 206 and execute such instructions to carry out computations and/or transfer data between various sources and destinations of data, such as hard disk 232, I/O processor 220, display interface 214, input devices 218, non-volatile memory 224, and the like.

Memory module 206 may include RAM (Random Access Memory), ROM (Read Only Memory), and other storage means, mapped to one addressable memory space. Memory module 206 illustrates one of many types of computer storage media for storage of information such as computer readable instructions, data structures, program modules or other data. Memory module 206 may store a basic input/output system (BIOS) for controlling low-level operation of computing device 200. Memory module 206 may also store OS 208 for controlling the general operation of computing device 200. It will be appreciated that OS 208 may include a general-purpose operating system such as a version of UNIX, or LINUX™, or a specialized client communication operating system such as Windows Mobile™, or the Symbian® operating system. OS 208 may, in turn, include or interface with a Java virtual machine (JVM) module that enables control of hardware components and/or operating system operations via Java application programs.

Memory module 206 may further include one or more distinct areas (by address space and/or other means), which can be utilized by computing device 200 to store, among other things, applications and/or other data. For example, one area of memory module 206 may be set aside and employed to store information that describes various capabilities of computing device 200, a device identifier, and the like. Such identification information may then be provided to another device based on any of a variety of events, including being sent as part of a header during a communication, sent upon request, or the like. One common software application is a browser program that is generally used to send/receive information to/from a web server. In one embodiment, the browser application is enabled to employ Handheld Device Markup Language (HDML), Wireless Markup Language (WML), WMLScript, JavaScript, Standard Generalized Markup Language (SMGL), HyperText Markup Language (HTML), eXtensible Markup Language (XML), and the like, to display and send a message. However, any of a variety of other web based languages may also be employed. In one embodiment, using the browser application, a user may view an article or other content on a web page with one or more highlighted portions as target objects.

Display interface 214 may be coupled with a display unit (not shown), such as liquid crystal display (LCD), gas plasma, light emitting diode (LED), or any other type of display unit that may be used with computing device 200. Display units coupled with display interface 214 may also include a touch sensitive screen arranged to receive input from an object such as a stylus or a digit from a human hand. Display interface 214 may further include interface for other visual status indicators, such Light Emitting Diodes (LED), light arrays, and the like. Display interface 214 may include both hardware and software components. For example, display interface 214 may include a graphic accelerator for rendering graphic-intensive outputs on the display unit. In one embodiment, display interface 214 may include software and/or firmware components that work in conjunction with CPU 204 to render graphic output on the display unit.

Audio interface 216 is arranged to produce and receive audio signals such as the sound of a human voice. For example, audio interface 216 may be coupled to a speaker and microphone (not shown) to enable communication with a human operator, such as spoken commands, and/or generate an audio acknowledgement for some action.

Input devices 218 may include a variety of device types arranged to receive input from a user, such as a keyboard, a keypad, a mouse, a touchpad, a touch-screen (described with respect to display interface 214), a multi-touch screen, a microphone for spoken command input (describe with respect to audio interface 216), and the like.

I/O processor 220 is generally employed to handle transactions and communications with peripheral devices such as mass storage, network, input devices, display, and the like, which couple computing device 200 with the external world. In small, low power computing devices, such as some mobile devices, functions of the I/O processor 220 may be integrated with CPU 204 to reduce hardware cost and complexity. In one embodiment, I/O processor 220 may the primary software interface with all other device and/or hardware interfaces, such as optical storage 202, hard disk 232, interfaces 226-228, display interface 214, audio interface 216, and input devices 218.

An electrical bus 222 internal to computing device 200 may be used to couple various other hardware components, such as CPU 204, memory module 206, I/O processor 220, and the like, to each other for transferring data, instructions, status, and other similar information.

Non-volatile memory 224 may include memory built into computing device 200, or portable storage medium, such as USB drives that may include PCM arrays, flash memory including NOR and NAND flash, pluggable hard drive, and the like. In one embodiment, portable storage medium may behave similarly to a disk drive. In another embodiment, portable storage medium may present an interface different than a disk drive, for example, a read-only interface used for loading/supplying data and/or software.

Various other interfaces 226-228 may include other electrical and/or optical interfaces for connecting to various hardware peripheral devices and networks, such as IEEE 1394 also known as FireWire, Universal Serial Bus (USB), Small Computer Serial Interface (SCSI), parallel printer interface, Universal Synchronous Asynchronous Receiver Transmitter (USART), Video Graphics Array (VGA), Super VGA (SVGA), HDMI (High Definition Multimedia Interface), and the like.

Network Interface Card (NIC) 230 may include circuitry for coupling computing device 200 to one or more networks, and is generally constructed for use with one or more communication protocols and technologies including, but not limited to, Global System for Mobile communication (GSM), code division multiple access (CDMA), time division multiple access (TDMA), user datagram protocol (UDP), transmission control protocol/Internet protocol (TCP/IP), SMS, general packet radio service (GPRS), WAP, ultra wide band (UWB), IEEE 802.16 Worldwide Interoperability for Microwave Access (WiMax), SIP/RTP, Bluetooth, Wi-Fi, Zigbee, UMTS, HSDPA, WCDMA, WEDGE, or any of a variety of other wired and/or wireless communication protocols.

Hard disk 232 is generally used as a mass storage device for computing device 200. In one embodiment, hard disk 232 may be a Ferro-magnetic stack of one or more disks forming a disk drive embedded in or coupled to computing device 200. In another embodiment, hard drive 232 may be implemented as a solid-state device configured to behave as a disk drive, such as a flash-based hard drive. In yet another embodiment, hard drive 232 may be a remote storage accessible over network interface 230 or another interface 226, but acting as a local hard drive. Those skilled in the art will appreciate that other technologies and configurations may be used to present a hard drive interface and functionality to computing device 200 without departing from the spirit of the present disclosure.

Power supply 234 provides power to computing device 200. A rechargeable or non-rechargeable battery may be used to provide power. The power may also be provided by an external power source, such as an AC adapter or a powered docking cradle that supplements and/or recharges a battery.

Transceiver 236 generally represents transmitter/receiver circuits for wired and/or wireless transmission and receipt of electronic data. Transceiver 236 may be a stand-alone module or be integrated with other modules, such as NIC 230. Transceiver 236 may be coupled with one or more antennas for wireless transmission of information.

Antenna 238 is generally used for wireless transmission of information, for example, in conjunction with transceiver 236, NIC 230, and/or GPS 242. Antenna 238 may represent one or more different antennas that may be coupled with different devices and tuned to different carrier frequencies configured to communicate using corresponding protocols and/or networks. Antenna 238 may be of various types, such as omni-directional, dipole, slot, helical, and the like.

Haptic interface 240 is configured to provide tactile feedback to a user of computing device 200. For example, the haptic interface may be employed to vibrate computing device 200, or an input device coupled to computing device 200, such as a game controller, in a particular way when an event occurs, such as hitting an object with a car in a video game.

Global Positioning System (GPS) unit 242 can determine the physical coordinates of computing device 200 on the surface of the Earth, which typically outputs a location as latitude and longitude values. GPS unit 242 can also employ other geo-positioning mechanisms, including, but not limited to, triangulation, assisted GPS (AGPS), E-OTD, CI, SAI, ETA, BSS or the like, to further determine the physical location of computing device 200 on the surface of the Earth. It is understood that under different conditions, GPS unit 242 can determine a physical location within millimeters for computing device 200. In other cases, the determined physical location may be less precise, such as within a meter or significantly greater distances. In one embodiment, however, a mobile device represented by computing device 200 may, through other components, provide other information that may be employed to determine a physical location of the device, including for example, a MAC address.

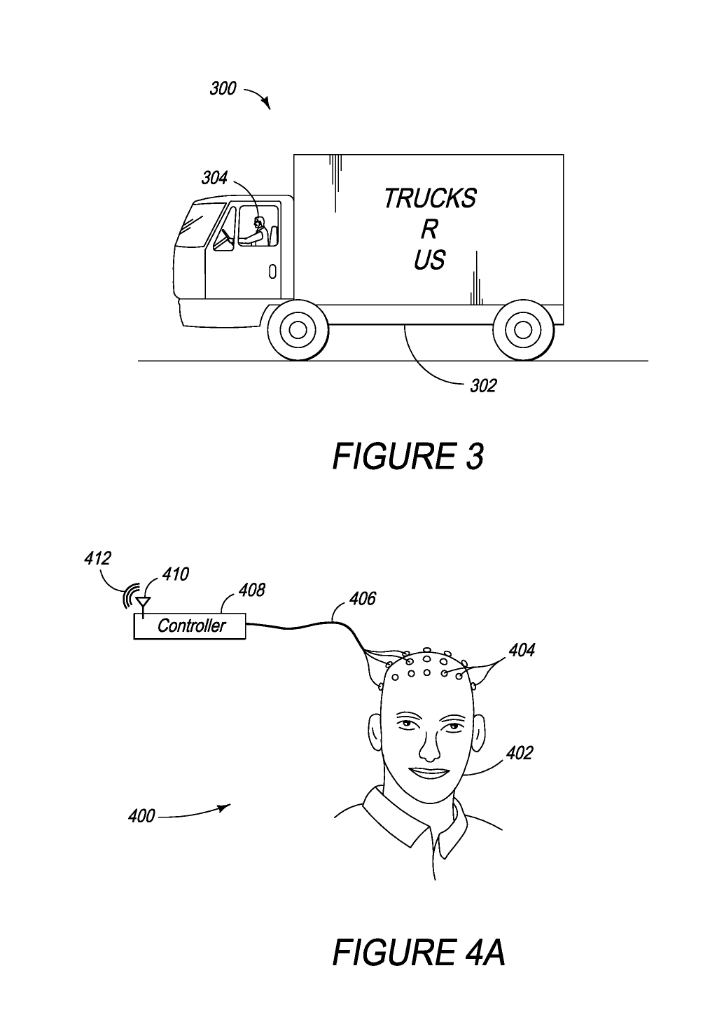

FIG. 3 shows an example operating environment wherein the disclosure may be practiced. In one embodiment, in vehicle arrangement 300, truck 302 is driven by user 304. Generally, driving a vehicle or operating other machinery safely is conditioned upon the mental alertness of the operator. During long and uneventful drives, the driver of a vehicle can easily fall asleep behind the steering wheel causing an accident, which may result in property damage, great bodily injury, and/or death.

A method and/or system to alert the driver in case he begins to fall asleep is highly desirable. However, detecting the operator's behavior, such as slumping over the wheel, hands falling off a sensor, or detecting other abnormal body posture, may be too late to prevent an accident. If an upcoming or imminent event, such as falling asleep, is detected prior to a corresponding external behavior of the user's body associated with the event, then an alert may be issued to the user to prevent the undesirable event.

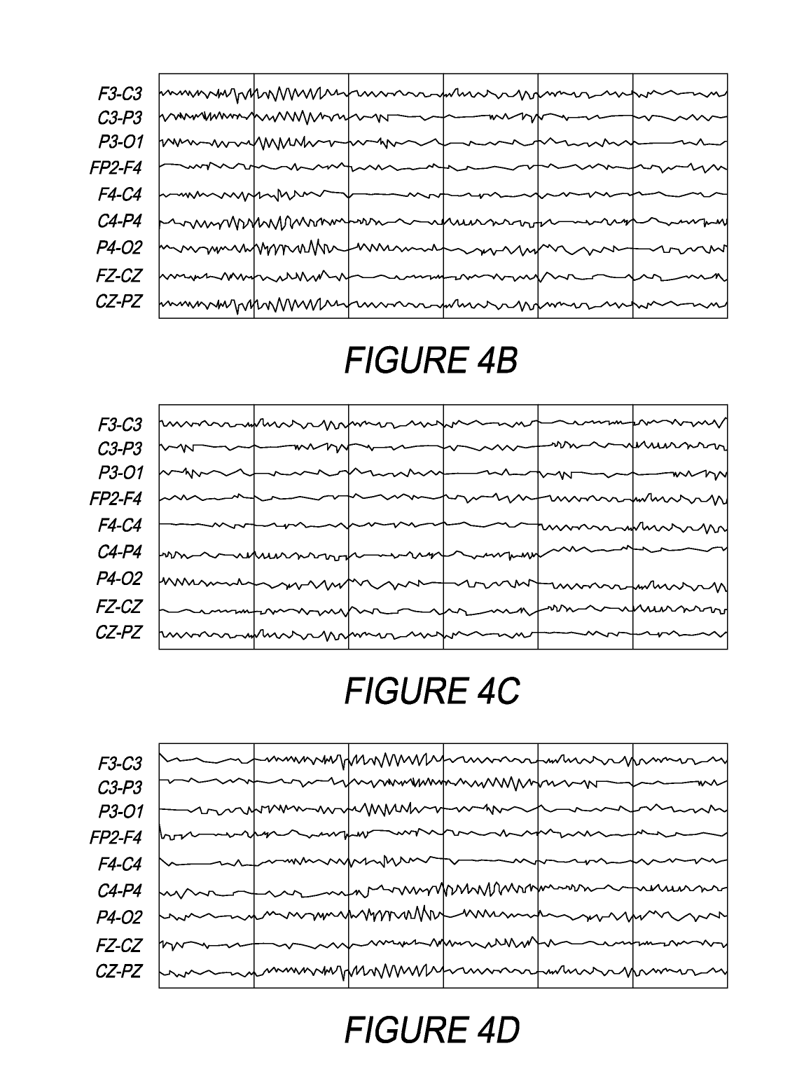

FIG. 4A shows an example arrangement for recording brainwave patterns. In various embodiments, EEG recording arrangement 400 includes one or more electrodes 404 attached to the scalp of user 402, and coupled to controller 408 via wires 406. Controller 408 may include antenna 410 to transmit wireless data 412.

In various embodiments, electrodes 404 are arranged at certain distance from each other and a ground electrode to measure the voltage on the scalp at the corresponding point with respect to the ground electrode or with respect to each other. The electrical signals, representing brainwaves, detected by electrodes 404 are transmitted to controller 408 via wires 406. In some embodiments, the detected signals are printed by the controller on paper, while in other embodiments, the data are stored on the controller itself for later printing or transmission to another location. In various embodiments, controller 408 transmits the obtained brainwave signals, by wire or wirelessly via antenna 410, to a central computer or database for storage and/or further analysis.

In various embodiments, controller 408 controls activation of electrodes 404, collects and/or stores data generated by the electrodes, analyzes signal patterns from individual electrodes and/or predefined subsets of all the electrodes, identifies an event associated with a particular signal pattern, and generates an alarm to prevent or otherwise manage the event.

In various embodiments, in addition to generating an alarm to alert the user/operator of an upcoming and highly probable or imminent event, such as falling asleep, in some embodiments, the controller sends the collected data and/or analysis results to a central office or computer for analysis and pattern recognition, identification of brain condition associated with the brainwave pattern, records keeping, legal purposes, insurance purposes, performance review, training, statistical analysis, policy setting, work environment improvement, and the like. In some embodiments, the central computer may take an action in response to detecting a particular brain state based on the transmitted data, including sending a remote alarm to the user.

Such data and analysis results may aid in litigation by providing evidence regarding the condition of the operator at time of an accident; may be helpful in setting insurance rates; may be used for performance review and/or training of operators; may be used for aggregate statistical analysis, for example, to ascertain productivity or optimize human resource allocation; may be used to set work policy such as maximum number of continuous work hours in a job function; and may also be used to improve work environment, for example, by adjusting lighting, color schemes, sounds, ergonomic aspects of workplace, and the like to reduce probability of undesirable events, such as falling asleep. Such data may also be used in real time to reschedule tasks based on driver/operator condition, for example, using real-time data indicating one worker may need rest and to deploy another worker for a job or delivery.

In various embodiments, the alarm system and method may be useful for truck drivers, automobile drivers, air traffic control officers, and others employed in hazardous, monotonous jobs where falling asleep could cause accidents to the operator, error in production, and lost productivity. The insurance rates may also be decreased as a result of using such preventive measures, which detect an event, such as falling asleep, based on brainwave patterns prior to the occurrence of the event and reduce risk. The alarm system and method may also be useful for epileptic, suicidal, or depressed patients or people with other similar medical conditions to alert them of an impending epileptic attack to sit down and avoid falling and being injured.

In various embodiments, the brainwave data are collected every few minutes, such as every five minutes, while in other embodiments, the frequency of measurement and analysis is adjustable by the user, the controller, or the central computer. In some embodiments, the measurement may continuous.

In various embodiments, the alarm generated may be in the form of sound, a mild electric shock, a motion such as vibration of a seat, providing extra oxygen to the user, other external physical stimulations, or any combination thereof which may serve to alert the user to take appropriate action. For example, if a truck driver is detected to be falling asleep, a loud beep may be played and extra oxygen provided to the passenger cabin to help prevent him from falling asleep or wake him up quickly if he has fallen asleep.

FIG. 4B-4D show examples of brainwave patterns associated with successive pre-sleep states. Upon detection of the signals, the temporal patterns (signal of one electrode over time) as well as spatial patterns (signals of different electrodes at one time) may be analyzed and compared with reference patterns to identify or classify the pattern and its associated brain state. Those skilled in the art will appreciate that there are many pattern recognition and classification algorithms available for identifying and/or classifying patterns. For example, features extraction and comparison may be employed, which extracts certain features out of a signal pattern, such as slope of variations, number of peaks and valleys per unit time, normalized signal power level or energy, and the like for comparison with predefined criteria or thresholds to classify the pattern.

FIG. 4B shows an example brainwave associated with the earliest indication of transition from wakefulness to stage I sleep (drowsiness), which usually consists of a combination of (1) drop out of alpha activity of brain and (2) slow rolling eye movements.

FIG. 4C shows an example EEG associated with slow lateral rolling eye movements during stage I sleep. Like faster lateral eye movements, slow ones are best seen at the F7 and F8 electrodes and corresponding signal lines.

FIG. 4D shows an example EEG with signals having typical vertex sharp transients. These patterns are narrow (brief) and more focal and are seen in sleep stages I and II.

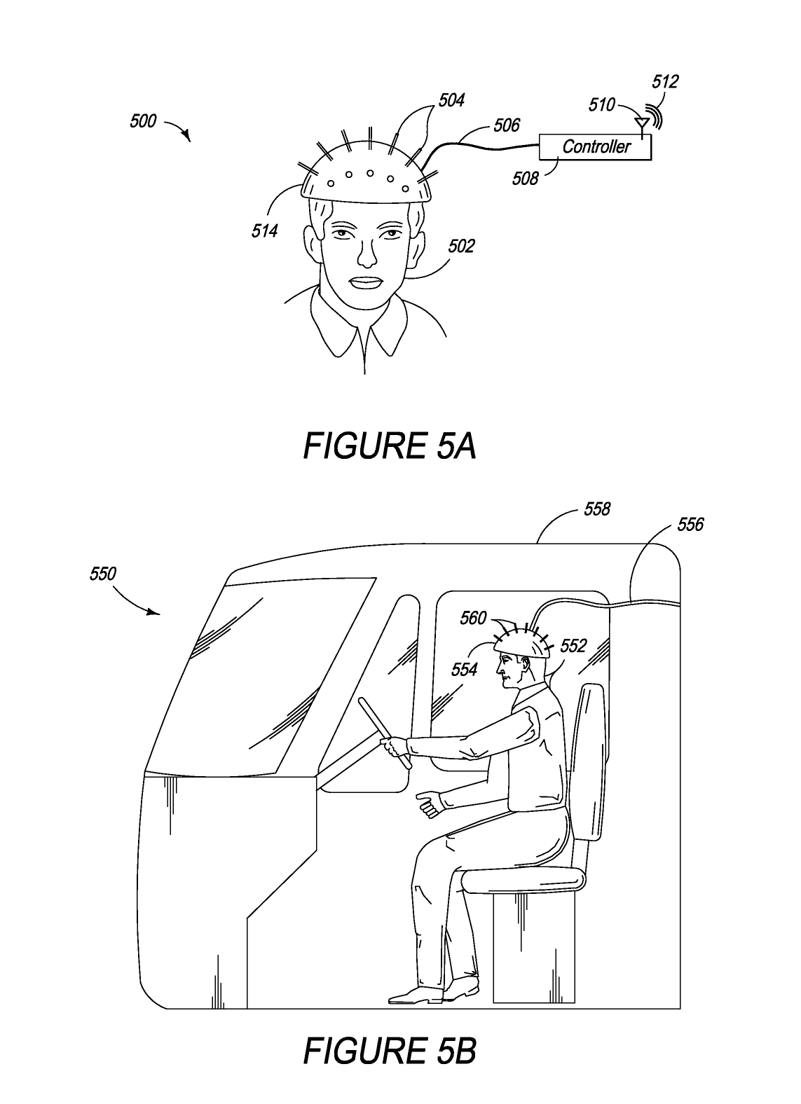

FIG. 5A shows an example brainwave detection cap and controller. In various embodiments, arrangement 500 includes user 502 using electrodes 504 embedded in electrode harness 514 and coupled to controller 508 with wires 506. Controller 508 may include antenna 510 for transmitting wireless signals 512 to a central computer for processing.

In various embodiments, electrode harness 514 may be a cap, a helmet, a mesh, a headband, a hairpiece, a specialized bowl or cavity coupled to a surrounding structure, and the like or even a band of data gathering sensors or a single point of contact with the head or a single point of data gathering proximate to the head. The electrodes may be small in size and weight to prevent interference with the normal operation of user 502. In some embodiments, electrodes 504 may be camouflaged as patterns on a cap to improve appearance and reduce distraction in a normal working environment. In various embodiments, electrode harness is a sporting helmet, for example a football helmet, configured to detect concussions in a sporting event.

FIG. 5B shows an example brainwave detection cap installed in the cab of a vehicle. In various embodiments, driving arrangement 550 includes user 552 sitting in vehicle cab 558 having an integrated electrode harness 554 with embedded electrodes 560 coupled to cab 558 using arm 556.

In various embodiments, electrode harness 554 is coupled to cab 558 using repositionable arm 556 to place the electrodes in close proximity of user 552's scalp to detect the user's brainwave patterns during driving. In some embodiments, the electrode harness operates in a manner similar to safety seat belts which reminds, using a buzzer, for example, the user to wear it or place it on his head while driving, while in other embodiments it is optional and the user may choose to use it for his own safety. In some embodiments, the use of the electrode harness is reported, via a wireless connection, to a central control office of a professional transportation business, such as a taxi or truck transport company, which may take appropriate action. For example, if a truck driver or a taxi driver is required by the company to wear the electrode harness but fails to do so, the central office may disable the vehicle remotely, using a processor on board of the vehicle, and not allow it to drive.

Those skilled in the art will appreciate that the electrode harness and the corresponding method of brain state detection, using EEG or other similar or derivative techniques, may be used to control operation of commercial machinery and vehicles in many ways without departing from the spirit of the present disclosures. For example, such devices and methods of alerting a user or a supervisor may be used in night shifts at factories, on commercial passenger vehicles, ships, and the like.

It will be understood that each block of the flowchart illustration, and combinations of blocks in the flowchart illustration, can be implemented by computer program instructions. These program instructions may be provided to a processor to produce a machine, such that the instructions, which execute on the processor, create means for implementing the actions specified in the flowchart block or blocks. The computer program instructions may be executed by a processor to cause a series of operational steps to be performed by the processor to produce a computer implemented process such that the instructions, which execute on the processor to provide steps for implementing the actions specified in the flowchart block or blocks. The computer program instructions may also cause at least some of the operational steps shown in the blocks of the flowchart to be performed in parallel. Moreover, some of the steps may also be performed across more than one processor, such as might arise in a multi-processor computer system. In addition, one or more blocks or combinations of blocks in the flowchart illustration may also be performed concurrently with other blocks or combinations of blocks, or even in a different sequence than illustrated without departing from the scope or spirit of the disclosure.

Accordingly, blocks of the flowchart illustration support combinations of means for performing the specified actions, combinations of steps for performing the specified actions and program instruction means for performing the specified actions. It will also be understood that each block of the flowchart illustration, and combinations of blocks in the flowchart illustration, can be implemented by special purpose hardware based systems which perform the specified actions or steps, or combinations of special purpose hardware and computer instructions.

Changes can be made to the claimed invention in light of the above Detailed Description. While the above description details certain embodiments of the invention and describes the best mode contemplated, no matter how detailed the above appears in text, the claimed invention can be practiced in many ways. Details of the system may vary considerably in its implementation details, while still being encompassed by the claimed invention disclosed herein.

Particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the claimed invention to the specific embodiments disclosed in the specification, unless the above Detailed Description section explicitly defines such terms. Accordingly, the actual scope of the claimed invention encompasses not only the disclosed embodiments, but also all equivalent ways of practicing or implementing the claimed invention.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The above specification, examples, and data provide a complete description of the manufacture and use of the claimed invention. Since many embodiments of the claimed invention can be made without departing from the spirit and scope of the disclosure, the invention resides in the claims hereinafter appended. It is further understood that this disclosure is not limited to the disclosed embodiments, but is intended to cover various arrangements included within the spirit and scope of the broadest interpretation so as to encompass all such modifications and equivalent arrangements.

What is claimed is:

1. A device for generating an alarm, the device comprising:
an electrode harness;
at least one electrode coupled with the electrode harness; and
a controller coupled with the at least one electrode configured to receive at least one signal from the at least one electrode, and further configured to generate an alarm in response to detecting a brain state.

2. The device of claim 1, further comprising an antenna coupled with the controller and configured to transmit data from the controller to a central computer.

3. The device of claim 1, wherein the electrode harness is one of a cap, a mesh, and a headband.

4. The device of claim 1, wherein the controller is further configured to analyze the at least one signal.

5. The device of claim 1, wherein the controller is further configured to wirelessly transmit the at least one signal to a central computer.

6. The device of claim 1, wherein the alarm is one or more of a sound, a vibration, and an electric shock delivered to a user.

7. The device of claim 1, wherein the brain state is a pre-sleep state of a user's brain.

8. The device of claim 1, wherein the brain state is associated with a pattern specified by a combination of the signals.

9. The device of claim 1, wherein the brain state is associated with an inebriation.

10. A method of generating an alarm, the method comprising:
detecting a brain state based on at least one signal detected from the brain; and
generating an alarm to alert a user of the brain state.

11. The method of claim 10, further comprising analyzing the at least one signal detected from the brain to classify a signal pattern based on the at least one signal as associated with the brain state.

12. The method of claim 10, further comprising wirelessly transmitting data associated with the at least one signal to a central computer.

13. The method of claim 10, wherein the at least one signal is detected using at least one electrode placed on a user's scalp.

14. The method of claim 13, wherein the at least one electrode is embedded in one of a cap, a helmet, a mesh, and a headband.

15. The method of claim 10, wherein the brain event comprises a pre-sleep brain state.

16. The method of claim 10, wherein the alarm is one or more of a sound, a vibration, and an electric shock delivered to a user.

17. A method of controlling an operation of a machine, the method comprising:
coupling at least one electrode with a user's scalp to detect the user's brainwaves;
receiving at least one signal from the at least one electrode using a controller coupled with the at least one electrode; and
generating an alarm in response to detecting a brain state based on the user's brainwaves.

18. The method of claim 17, further comprising analyzing a user's brainwave patterns using the at least one signal to classify the user's brainwave patterns.

19. The method of claim 17, further comprising transmitting data based on the at least one signal to a central computer.

20. The method of claim 17, wherein the at least one electrode are embedded in a harness integrated with a vehicle.
